(19) United States
(12) Patent Application Publication
(10) Pub. No.: US 2008/0306039 A1
(43) Pub. Date: Dec. 11, 2008

(54) Loteprednol Etabonate Aqueous Suspension

(76) Inventor: Keiichi Matsuhisa, Hyogo (JP)

Correspondence Address: WENDEROTH, LIND & PONACK, L.L.P., 2033 K STREET N. W., SUITE 800, WASHINGTON, DC, 20006-1021, US

(21) Appl. No.: 10/581,244

(22) PCT Filed: Nov. 30, 2004
(86) PCT No.: PCT/JP2004/017762

§ 371 Date: May 31, 2006

(30) Foreign Application Priority Data
Dec. 2, 2003 (JP) 2003-403719

(51) Int. Cl. A61K 31/56

(52) U.S. Cl. 514/179

(57) ABSTRACT

An aqueous suspension containing loteprednol etabonate wherein sedimented particles are detached easily from the container and adhesion of sedimented particles to the container and block formation are inhibited so that redispersion has been improved is provided. A method for improving redispersibility of loteprednol etabonate contained in the aqueous suspension is also provided.

A loteprednol etabonate-containing aqueous suspension comprising loteprednol etabonate and at least one compound selected from the group consisting of sorbic acid, salts thereof, and p-hydroxybenzoate esters, and a non-ionic surfactant is prepared.

### TECHNICAL FIELD

The present invention relates to an aqueous suspension comprising loteprednol etabonate and at least one member selected from the group consisting of sorbic acid, salts thereof and p-hydroxybenzoic acid esters, and to a method for improvement of redispersibility of loteprednol etabonate contained in the aqueous suspension.

### BACKGROUND ART

Loteprednol etabonate (referred sometimes to as LE hereinafter) is a steroidal agent having anti-inflammatory action. LE, being a crystalline substance hardly soluble in water, need to be formed as a suspension to give an aqueous liquid preparation.

When an aqueous suspension is stored over a long time, particles of the agent contained in the suspension will form aggregates, or adhere or adsorb onto the wall of container, followed by secondary particle formation of the sedimented particles (block formation), so that redispersion may become difficult.

Aqueous suspensions with improved redispersibility reported so far include an aqueous suspension containing a water-soluble polymer within the concentration range from the concentration at which the surface tension of the liquid preparation begins to decrease up to the concentration at which the reduction in surface tension ceases and a hardly soluble agent (see Reference 1), an aqueous suspension-type eye-drop of a hardly soluble agent of a viscosity kept at 100 cP or less by incorporating an ionic polymer and a metal ion (see Reference 2), an aqueous suspension containing a hardly soluble agent, polyvinylpyrrolidone and a water-soluble anionic polymer (see Reference 3), and an aqueous suspension eye-drop of a hardly soluble agent containing a suspending agent selected from the group consisting of D-mannitol, D-sorbitol, xylitol, propyleneglycol and citrates, and mixtures thereof (see Reference 4).

LE-containing aqueous suspensions reported so far include a composition containing LE, a non-ionic polymer, a non-ionic surfactant, and a non-ionic isotonicity agent (see Reference 5), an aqueous suspension containing LE and an aliphatic amino acid having 2 to 7 carbons (see Reference 6), and an aqueous suspension for nasal-drop containing LE and crystalline cellulose/carmellose sodium (see Reference 7).

Reference 1: Japanese Unexamined Patent Publication No. H11-29463

Reference 2: Japanese Unexamined Patent Publication No. H08-295622

Reference 3: International Publication No. 02/15878 Pamphlet

Reference 4: Japanese Unexamined Patent Publication No. H10-36253

Reference 5: U.S. Pat. No. 5,540,930

Reference 6: Japanese Unexamined Patent Publication No. H10-316572

Reference 7: Japanese Unexamined Patent Publication No. H10-259132

### SUMMARY OF THE INVENTION

The invention provides an aqueous suspension comprising LE and at least one member selected from the group consisting of sorbic acid, salts thereof and p-hydroxybenzoic acid esters. The invention provides also a method for improvement of redispersibility of LE contained in the aqueous suspension.

As the result of the inventors' intensive researches to attain the above-mentioned objective, the inventors have found that by compounding at least one member selected from the group consisting of sorbic acid, salts thereof, and p-hydroxybenzoic acid esters in an LE-containing aqueous suspension, adhesion of sedimented LE particles to the container and block formation are prevented so that redispersibility is improved, and as the results of further researches based on this finding the inventors have finally completed the present invention.

Namely, the invention relates to:

(1) An aqueous suspension comprising loteprednol etabonate and at least one member selected from the group consisting of sorbic acid, salts thereof and p-hydroxybenzoic acid esters,

(2) An aqueous suspension as described in (1) that contains additionally a non-ionic surfactant,

(3) An aqueous suspension as described in (1) or (2) that is an eye-drop,

(4) An aqueous suspension as described in (1) or (2) that is a nasal-drop,

(5) An aqueous suspension as described in (1) or (2) that is an ear-drop, and

(6) A method for improving redispersibility of loteprednol etabonate which comprises compounding at least one member selected from the group consisting of sorbic acid, salts thereof and p-hydroxybenzoic acid esters in an aqueous suspension containing loteprednol etabonate

### EFFECT OF THE INVENTION

According to the invention, by compounding at least one compound selected from the group consisting of sorbic acid, salts thereof, and p-hydroxybenzoate esters in an aqueous suspension containing LE, adhesion of sedimented LE particles to the container and block formation are inhibited so that an aqueous suspension containing LE with improved redispersibility may be provided.

### DETAILED DESCRIPTION OF THE PREFERRED EXAMPLES

The invention is explained in more detail in the following.

The concentration of LE in the aqueous suspension of the invention may be any concentration as far as the suspension is therapeutically effective against inflammation, the lower limit of the concentration being usually about 0.01 w/v %, desirably about 0.05 w/v %, and more desirably about 0.1 w/v % and the upper limit of the concentration being usually about 2.0 w/v %, desirably about 1.5 w/v %, and more desirably about 1.0 w/v %.

Sorbic acid and salts thereof used in the invention include sorbic acid, potassium sorbate, and sodium sorbate. Potassium sorbate is preferable.

The concentration of sorbic acid or its salt in the aqueous suspension of the invention is not specified though the lower limit of the concentration is usually about 0.001 w/v % and desirably about 0.005 w/v %, and the upper limit of the concentration is usually about 5.0 w/v % and desirably about 1.0 w/v %.

The p-hydroxybenzoic acid esters used in the invention are desirably products esterified with a lower alkyl group, including methyl p-hydroxybenzoate, ethyl p-hydroxybenzoate, propyl p-hydroxybenzoate, isopropyl p-hydroxybenzoate, butyl p-hydroxybenzoate, and isobutyl p-hydroxybenzoate. The concentration of the p-hydroxybenzoate ester in the aqueous suspension of the invention is not specified though the lower limit is usually about 0.001 w/v % and desirably about 0.01 w/v %, and the upper limit is usually about 1.0 w/v % and desirably about 0.1 w/v %.

The above-mentioned sorbic acid, salts thereof, and p-hydroxybenzoate esters may be used individually or in combination of two or more of them.

For manufacturing of the aqueous suspension of the invention, is used a non-ionic surfactant. The non-ionic surfactants used include tyloxapol, polysorbate 80, and polypropylene glycol-ethylenoxide block polymer. Tyloxapol is preferable. The concentration of the non-ionic surfactant in the aqueous suspension of the invention is not particularly limited though the lower limit is usually about 0.01 w/v % and desirably about 0.05 w/v % and the upper limit is usually about 5.0 w/v % and desirably about 1.0 w/v %.

To the aqueous suspension of the invention, may be added appropriately an isotonicity agent (sodium chloride, potassium chloride, glycerol, mannitol, sorbitol, propylene glycol, boric acid, etc.), a buffering agent (phosphate buffer, acetate buffer, borate buffer, carbonate buffer, citrate buffer, Tris buffer, glutamic acid, ε-aminocapronic acid, sodium acetate, boric acid, borax, etc.), a preservative (chlorobutanol, benzyl alcohol, sodium dehydroacetate, sodium edetate, benzalkonium chloride, benzethonium chloride, boric acid, borax, etc.), a water-soluble polymer (hydroxyethylcellulose, hydroxypropylcellulose, hydroxypropylmethylcellulose, polyvinyl alcohol, polyvinylpyrrolidone, etc.), a stabilizer (sodium bisulfite, sodium thiosulfate, sodium edetate, sodium citrate, sodium acetate, ascorbic acid, dibutyl hydroxy toluene, boric acid, borax, etc.), a pH-adjusting agent (hydrochloric acid, sodium hydroxide, phosphoric acid, acetic acid, etc.), a refrigerant (camphor, menthol, etc.), etc.

The amount of the additive may vary according to the type, the purpose of use, etc. of the additive and an additive is added to the concentration at which the purpose of the additive is attained. For example, an isotonicity agent is usually added to the concentration of about 0.5 to about 5.0 w/v % so that the osmotic pressure may become about 229 to about 343 mOsm. A buffering agent is added to the concentration of about 0.01 to about 2.0 w/v %, a water-soluble polymer to the concentration of about 0.0001 to about 2.0 w/v %, and a stabilizer to the concentration of about 0.001 to about 1.0 w/v %. A pH-adjusting agent is added appropriately so that pH may be adjusted usually to about 4.0 to about 9.0, and preferably to about 5.0 to about 8.0. A preservative is added to the concentration of about 0.001 to about 3.0 w/v %.

In the aqueous suspension of the invention, may be compounded a medicinal ingredient other than LE, such as a therapeutic agent for glaucoma, a steroidal or non-steroidal anti-inflammatory agent, an antimicrobial agent, an angiotonic, an anti-allergic agent, an anti-histamic agent, an anti-viral agent, etc. as far as the ingredient is not against the objective of the invention.

The aqueous suspension of the invention, being excellent in redispersion, can be used as a drug (for example, for prevention or treatment of allergy and inflammation) in human or as an animal drug in mammals other than human (for example, rat, mouse, guinea pig, monkey, dog, cow, pig, etc.). The aqueous suspension of the invention can be utilized favorably as eye-drops, nasal-drops, ear-drops, injections, liquids for internal use, liniments, and lotions, among which use as eye-drops, nasal-drops and ear-drops is desirable. For example, when the aqueous suspension of the invention is used for treatment of inflammation of eyes suffering from conjunctivitis, blepharitis, keratitis, seleritis, iritis, iridocyclitis, uveitis, postoperative inflammation, allergic conjunctivitis, trachoma, etc., an eye-drop containing LE at 0.5 w/v % is applied at the dose of one or two drops on one occasion 3 to 5 times a day in an adult.

The aqueous suspension of the invention can be manufactured by a publicly known method for preparation, for example a method described in Liquids, Suspensions, and Ophthalmic Solutions, General Rules for Preparations, JP XIV.

### EXAMPLES

The invention is illustrated in further details below on the basis of Examples and Test Examples, which however do not limit the invention.

### Test Example 1

### Redispersion Test

### Test Method

The LE-containing aqueous suspensions shown in Table 1 were prepared by the conventional method and each suspension was filled in polypropylene (PP) containers in 5 mL each (n=4). The containers were kept upright at 4° C. for 1 week to allow LE particles to sediment. Then the containers were turned upside down and kept at 40° C. and 75% RH for 1 week to allow LE particles to adhere to the container. After storage at 4° C. for 1 week and at 40° C. for 1 week, the containers were shaken 20 times, and the sediment adhered to the container and aggregates (block) formed after detachment of the sediment were observed visually. Using the LE-containing aqueous suspension in a container kept upright at 4° C. for 2 weeks, the number of shaking times required for detachment of the sediment from the container was determined.

TABLE 1
Preparation for LE-containing aqueous suspension

| Preparation No. | S-0 | | S-1 | | S-2 | | S-3 | |
|---|---|---|---|---|---|---|---|---|
| Loteprednol etabonate | 0.5 | g | 0.5 | g | 0.5 | g | 0.5 | g |
| Potassium sorbate | — | | 0.01 | g | 0.05 | g | 0.2 | g |
| Tyloxapol | 0.2 | g | 0.2 | g | 0.2 | g | 0.2 | g |
| ε-Aminocapronic acid | 0.2 | g | 0.2 | g | 0.2 | g | 0.2 | g |
| Sodium chloride | 0.75 | g | 0.75 | g | 0.75 | g | 0.75 | g |
| Sodium edetate | 0.01 | g | 0.01 | g | 0.01 | g | 0.01 | g |
| Benzalkonium chloride | 0.005 | g | 0.005 | g | 0.005 | g | 0.005 | g |
| Hydrochloric acid | Appropriate | | Appropriate | | Appropriate | | Appropriate | |
| | quantity | | quantity | | quantity | | quantity | |
| Sterile purified water | Appropriate | | Appropriate | | Appropriate | | Appropriate | |
| | quantity | | quantity | | quantity | | quantity | |
| Total volume | 100 | mL | 100 | mL | 100 | mL | 100 | mL |

| | | | | |
|---|---|---|---|---|
| pH | 5.5 | 5.5 | 5.5 | 5.5 |

### Test Results

The results of the test are shown in Table 2.

TABLE 2
Results of redispersion test of LE-containing aqueous suspension

| Preparation No. | S-0 | S-1 | S-2 | S-3 |
|---|---|---|---|---|
| Adhesion * | 1/4 | 0/4 | 1/4 | 1/4 |
| Block ** | 3/4 | 0/4 | 0/4 | 1/4 |
| Number of shaking times required | 9 | 5 | 5 | 7 |
| for detachment after storage at | | | | |
| 4° C. for 2 weeks *** | | | | |
| (mean for n = 4) | | | | |

* (number of samples where substance adhered to the container was found after 20 times of shaking)/(number of samples tested)
** (number of samples where block was noted after 20 times of shaking)/(number of samples tested)
*** number of times of shaking required for detachment of the sediment from the container using the LE-containing aqueous suspension in the container kept upright at 4° C. for 2 weeks

As seen from the above-mentioned test results, the LE-containing aqueous suspension without potassium sorbate (S-0) showed adhesion of sedimented LE particles to the container and formation of block. In contrast, LE-containing aqueous suspensions with potassium sorbate (S-1, S-2, S-3) had decreased frequency of adhesion of sedimented LE particles to the container and formation of block after detachment. LE-containing aqueous suspensions with potassium sorbate (S-1, S-2, S-3) after storage at 4° C. for 2 weeks showed reduced number of times of shaking required for detachment of the sediment from the container.

These results indicate that compounding of a sorbate in the LE-containing aqueous suspension inhibited adhesion of sedimented LE particles to the container and block formation after detachment from the container, and made detachment of sedimented LE particles easier, so that redispersibility was improved.

### Test Example 2

### Redispersion Test

### Test Method

The LE-containing aqueous suspensions shown in Table 3 were prepared by the conventional method and each suspension was filled in polypropylene (PP) containers in 5 mL each (n=8). The containers were kept upright at 4° C. for 1 week to allow LE particles to sediment. Then the containers were turned upside down and kept at 40° C. and 75% RH for 1 week to allow LE particles to adhere to the bottom of the container. After storage at 4° C. for 1 week and at 40° C. for 1 week, each container was shaken, and the number of shaking times required for detachment of the sediment from the container was determined.

TABLE 3
Preparation for LE-containing aqueous suspension

| Preparation No. | P-0 | | P-1 | | P-2 | |
|---|---|---|---|---|---|---|
| Loteprednol etabonate | 0.5 | g | 0.5 | g | 0.5 | g |
| Potassium sorbate | — | | 0.1 | g | — | |
| Methyl p-hydroxybenzoate | — | | — | | 0.026 | g |
| Propyl p-hydroxybenzoate | — | | — | | 0.014 | g |
| Tyloxapol | 0.2 | g | 0.2 | g | 0.2 | g |
| ε-Aminocapronic acid | 0.2 | g | 0.2 | g | 0.2 | g |
| Concentrated glycerin | 2.6 | g | — | | 2.6 | g |
| Boric acid | — | | 1.5 | g | — | |
| Sodium edetate | 0.01 | g | 0.01 | g | 0.01 | g |
| Benzalkonium chloride | 0.005 | g | — | | — | |
| Hydrochloric acid | Appropriate | | Appropriate | | Appropriate | |
| | quantity | | quantity | | quantity | |
| Sterile purified water | Appropriate | | Appropriate | | Appropriate | |
| | quantity | | quantity | | quantity | |
| Total volume | 100 | mL | 100 | mL | 100 | mL |

| | | | |
|---|---|---|---|
| pH | 5.5 | 5.5 | 5.5 |

### Test Results

The results of the test are shown in Table 4.

TABLE 4
Results of redispersion test of LE-containing aqueous suspension

| Preparation No. | P-0 | P-1 | P-2 |
|---|---|---|---|
| Number of shaking times | 19 | 13 | 5 |
| required for detachment | | | |
| (mean for n = 8) | | | |

As seen from the above-mentioned test results, the number of shaking times required for detachment of the sediment was 19 for the LE-containing aqueous suspension without potassium sorbate (P-0) and any p-hydroxybenzoate ester whereas the number was reduced to 13 for the LE-containing aqueous suspension with potassium sorbate (P-1) and to 5 for the LE-containing aqueous suspension with a p-hydroxybenzoate ester (P-2).

These results indicate that compounding of a sorbate or a p-hydroxybenzoate ester in the LE-containing aqueous suspension made detachment of sedimented LE particles from the container easier, so that redispersion was improved.

### Preparation Example 1

### Aqueous Suspension for Eye-Drop

| | | |
|---|---|---|
| Loteprednol etabonate | 0.5 | g |
| Potassium sorbate | 0.2 | g |
| Tyloxapol | 0.2 | g |
| ε-Aminocapronic acid | 0.2 | g |
| Sodium chloride | 0.75 | g |
| Sodium edetate | 0.01 | g |
| Benzalkonium chloride | 0.005 | g |

| | |
|---|---|
| Hydrochloric acid | appropriate quantity |
| Sterile purified water | to make a total volume of 100 mL |
| pH | 5.5 |

According to the above preparation, potassium sorbate, tyloxapol, ε-aminocapronic acid, sodium chloride, sodium edetate, and benzalkonium chloride were added to and dissolved in about 80 mL of sterile purified water. Loteprednol etabonate was added and suspended homogenously using a homogenizer, and pH was adjusted to 5.5 by addition of hydrochloric acid. Sterile purified water was added to make a total volume of 100 mL to prepare an aqueous suspension eye-drop containing loteprednol etabonate.

### Preparation Example 2

### Aqueous Suspension for Eye-Drop

| | | |
|---|---|---|
| Loteprednol etabonate | 0.5 | g |
| Methyl p-hydroxybenzoate | 0.026 | g |
| Propyl p-hydroxybenzoate | 0.014 | g |
| Tyloxapol | 0.2 | g |
| ε-Aminocapronic acid | 0.2 | g |
| Concentrated glycerin | 2.6 | g |
| Sodium edetate | 0.01 | g |

| | |
|---|---|
| Hydrochloric acid | appropriate quantity |
| Sterile purified water | to make a total volume of 100 mL |
| pH | 5.5 |

According to the above preparation, methyl p-hydroxybenzoate, propyl p-hydroxybenzoate, tyloxapol, ε-aminocapronic acid, concentrated glycerin, and sodium edetate were added to and dissolved in about 80 mL of sterile purified water. Loteprednol etabonate was added and suspended homogenously using a homogenizer, and pH was adjusted to 5.5 by addition of hydrochloric acid. Sterile purified water was added to make a total volume of 100 mL to prepare an aqueous suspension eye-drop containing loteprednol etabonate.

### Preparation Example 3

### Aqueous Suspension for Eye-Drop

| | | |
|---|---|---|
| Loteprednol etabonate | 0.5 | g |
| Methyl p-hydroxybenzoate | 0.026 | g |
| Propyl p-hydroxybenzoate | 0.014 | g |
| Tyloxapol | 0.3 | g |
| Chlorobutanol | 0.3 | g |
| ε-Aminocapronic acid | 0.2 | g |
| Concentrated glycerin | 2.6 | g |
| Polyvinylpyrrolidone K-30 | 0.6 | g |
| Sodium edetate | 0.01 | g |

| | |
|---|---|
| Hydrochloric acid | appropriate quantity |
| Sterile purified water | to make a total volume of 100 mL |
| pH | 5.5 |

According to the above preparation, methyl p-hydroxybenzoate, propyl p-hydroxybenzoate, tyloxapol, chlorobutanol, ε-aminocapronic acid, concentrated glycerin, polyvinylpyrrolidone K-30, and sodium edetate were added to and dissolved in about 80 mL of sterile purified water. Loteprednol etabonate was added and suspended homogenously using a homogenizer, and pH was adjusted to 5.5 by addition of hydrochloric acid. Sterile purified water was added to make a total volume of 100 mL to prepare an aqueous suspension eye-drop containing loteprednol etabonate.

### Preparation Example 4

### Aqueous Suspension for Eye-Drop

| | | |
|---|---|---|
| Loteprednol etabonate | 0.5 | g |
| Methyl p-hydroxybenzoate | 0.026 | g |
| Propyl p-hydroxybenzoate | 0.014 | g |
| Tyloxapol | 0.3 | g |
| Chlorobutanol | 0.3 | g |
| ε-Aminocapronic acid | 0.1 | g |
| Concentrated glycerin | 2.6 | g |
| Sodium edetate | 0.01 | g |

| | |
|---|---|
| Hydrochloric acid | appropriate quantity |
| Sterile purified water | to make a total volume of 100 mL |
| pH | 5.5 |

According to the above preparation, methyl p-hydroxybenzoate, propyl p-hydroxybenzoate, tyloxapol, chlorobutanol, ε-aminocapronic acid, concentrated glycerin, and sodium edetate were added to and dissolved in about 80 mL of sterile purified water. Loteprednol etabonate was added and suspended homogenously using a homogenizer, and pH was adjusted to 5.5 by addition of hydrochloric acid. Sterile purified water was added to make a total volume of 100 mL to prepare an aqueous suspension eye-drop containing loteprednol etabonate.

### Preparation Example 5

### Aqueous Suspension for Nasal-Drop

| | | |
|---|---|---|
| Loteprednol etabonate | 0.5 | g |
| Potassium sorbate | 0.2 | g |
| Tyloxapol | 0.2 | g |
| ε-Aminocapronic acid | 0.2 | g |
| Boric acid | 1.5 | g |
| Sodium edetate | 0.01 | g |
| Benzalkonium chloride | 0.005 | g |

| | |
|---|---|
| Hydrochloric acid | appropriate quantity |
| Sterile purified water | to make a total volume of 100 mL |
| pH | 5.5 |

According to the above preparation, potassium sorbate, tyloxapol, ε-aminocapronic acid, boric acid, sodium edetate, and benzalkonium chloride were added to and dissolved in about 80 mL of sterile purified water. Loteprednol etabonate was added and suspended homogenously using a homogenizer, and pH was adjusted to 5.5 by addition of hydrochloric acid. Sterile purified water was added to make a total volume of 100 mL to prepare an aqueous suspension nasal-drop containing loteprednol etabonate.

### Preparation Example 6

### Aqueous Suspension for Ear-Drop

| | | |
|---|---|---|
| Loteprednol etabonate | 0.5 | g |
| Methyl p-hydroxybenzoate | 0.026 | g |
| Propyl p-hydroxybenzoate | 0.014 | g |
| Tyloxapol | 0.3 | g |
| Chlorobutanol | 0.3 | g |
| ε-Aminocapronic acid | 0.2 | g |
| Concentrated glycerin | 2.6 | g |
| Sodium edetate | 0.01 | g |

| | |
|---|---|
| Hydrochloric acid | appropriate quantity |
| Sterile purified water | to make a total volume of 100 mL |
| pH | 5.5 |

According to the above preparation, methyl p-hydroxybenzoate, propyl p-hydroxybenzoate, tyloxapol, chlorobutanol, ε-aminocapronic acid, concentrated glycerin, and sodium edetate were added to and dissolved in about 80 mL of sterile purified water. Loteprednol etabonate was added and suspended homogenously using a homogenizer, and pH was adjusted to 5.5 by addition of hydrochloric acid. Sterile purified water was added to make a total volume of 100 mL to prepare an aqueous suspension ear-drop containing loteprednol etabonate.

### INDUSTRIAL APPLICABILITY

The invention can provide an aqueous suspension containing loteprednol eatabonate where adhesion of sedimented LE particles to the container and block formation are prevented and redispersibility has been improved as a result of compounding at least one member selected from the group consisting of sorbic acid, salts thereof and p-hydroxybenzoic acid esters in an aqueous suspension containing loteprednol etabonate. The aqueous suspension of the invention with good redispersibility can be utilized as an excellent eye-drop, nasal-drop, ear-drop so on.

1. An aqueous suspension comprising loteprednol etabonate and at least one member selected from the group consisting of sorbic acid, salts thereof and p-hydroxybenzoic acid esters.

2. The aqueous suspension according to claim 1 wherein a non-ionic surfactant is additionally contained.

3. The aqueous suspension according to claim 1 wherein the suspension is an eye-drop.

4. The aqueous suspension according to claim 1 wherein the suspension is a nasal-drop.

5. The aqueous suspension according to claim 1 wherein the suspension is an ear-drop.

6. A method for improving redispersibility of loteprednol etabonate which comprises compounding at least one member selected from the group consisting of sorbic acid, salts thereof and p-hydroxybenzoic acid esters in an aqueous suspension containing loteprednol etabonate.

7. The aqueous suspension according to claim 2 wherein the suspension is an eye-drop.

8. The aqueous suspension according to claim 2 wherein the suspension is a nasal-drop.

9. The aqueous suspension according to claim 2 wherein the suspension is an ear-drop.
